(19) United States
(12) Patent Application Publication
(10) Pub. No.: US 2022/0259557 A1
(43) Pub. Date: Aug. 18, 2022

(54) CELLS CAPABLE OF DIFFERENTIATING INTO PLACENTA-CONSTITUTING CELLS, AND METHOD FOR PRODUCING SAME

(71) Applicant: TOHOKU UNIVERSITY, Sendai-shi, Miyagi (JP)

(72) Inventors: Takahiro ARIMA, Sendai-shi, Miyagi (JP); Norio KOBAYASHI, Sendai-shi, Miyagi (JP); Hiroaki OKAE, Sendai-shi, Miyagi (JP)

(21) Appl. No.: 17/618,161

(22) PCT Filed: Jun. 14, 2019
(86) PCT No.: PCT/JP2019/023725

§ 371 Date: Dec. 10, 2021

(51) Int. Cl. C12N 5/073

(52) U.S. Cl. CPC C12N 5/0605; C12N 2501/115; C12N 2501/155; C12N 2533/90; C12N 2506/02; C12N 2506/45; C12N 2533/54; C12N 2501/727

(57) ABSTRACT

A cell derived from a pluripotent stem cell and that is capable of differentiating into a placenta-forming cell, and that is negative for BRDT, and positive for TP63. A method for producing a cell capable of differentiating into a placenta-forming cell, the method including the steps of: (a) culturing a pluripotent stem cell of a mammal with a medium containing bone morphogenetic protein 4; and (b) culturing the cell after the step (a) with a medium containing a growth factor and a ROCK inhibitor.

### TECHNICAL FIELD

The present invention relates to cells capable of differentiating into placenta-forming cells, and to a method for producing same.

### BACKGROUND ART

Trophoblasts are the main constituent of placental tissues. Trophoblast stem cells (TS cells) are considered highly beneficial for an understanding of the mechanism by which differentiation of trophoblast cells is regulated. To this end, there exists a need for establishment of a TS cell line that can be subcultured in a maintained undifferentiated state.

The present inventors have developed a method by which CD49f antibody positive cells collected from a cell suspension obtained from mammalian placental tissues are induced to differentiate into cells (TS-like cells) having similar characteristics to TS cells (PTL 1). However, the methods described in PTL 1 and NPL 1 require taking a cell suspension from placental tissues.

There are attempts to induce differentiation of ES cells into TS-like cells (NPL 2). However, the results of previous studies including analyses of gene expression pattern and DNA methylation pattern have revealed that many of these TS-like cells are not necessarily similar to TS cells.

### CITATION LIST

### Patent Literature

PTL 1: Japanese Patent No. 6400832

### Non Patent Literature

NPL 1: Okae H, et al., Derivation of Human Trophoblast Stem Cells. Cell Stem Cell. 2018 January; 22:50-63.
NPL 2: Gamage T K, et al., Stem cell insights into human trophoblast lineage differentiation. Hum Reprod Update. 2016 December; 23(1):77-103.

### SUMMARY OF INVENTION

### Technical Problem

As discussed above, previous attempts to induce differentiation of pluripotent stem cells into TS-like cells have been unsuccessful, and there is a need for development of a method that can induce differentiation of pluripotent stem cells into TS-like cells.

It is accordingly an object of the present invention to provide a method for producing TS-like cells from pluripotent stem cells. Another object is to provide TS-like cells produced by using the method.

### Solution to Problem

The present invention includes the following aspects.

[1] A cell derived from a pluripotent stem cell of a mammal and that is capable of differentiating into a placenta-forming cell, and that is negative for BRDT, and positive for TP63.

[2] The cell according to [1], which is negative for at least one selected from the group consisting of Oct4, Nanog, and Sox2.

[3] The cell according to [1] or [2], which is positive for at least one selected from the group consisting of GATA2, GATA3, and TFAP2A.

[4] The cell according to any one of [1] to [3], wherein a fraction of methylation of genomic DNA is 60% or less of whole genome.

[5] The cell according to any one of [1] to [4], wherein the placenta-forming cell is an extravillous cytotrophoblast or a syncytiotrophoblast.

[6] A method for producing a cell capable of differentiating into a placenta-forming cell,

the method including the steps of:

(a) culturing a pluripotent stem cell of a mammal with a medium containing bone morphogenetic protein 4 (BMP4); and

(b) culturing the cell after the step (a) with a medium containing a growth factor and a ROCK inhibitor.

[7] A method for producing a cell capable of differentiating into a placenta-forming cell,

the method including the steps of:

(a′) introducing at least one gene selected from the group consisting of GATA2, GATA3, and TFAP2A to a pluripotent stem cell of a mammal; and

(b) culturing the cell after the step (a′) with a medium containing a growth factor and a ROCK inhibitor.

[8] The method according to [6] or [7], wherein the medium in the step (b) further contains at least one selected from the group consisting of an ALK5 inhibitor and a GSK3β inhibitor.

### Advantageous Effects of Invention

The present invention can provide a method for producing TS-like cells from pluripotent stem cells. The present invention can also provide TS-like cells produced by using the method.

### BRIEF DESCRIPTION OF DRAWINGS

FIG. 1 shows a light micrograph of iPS cells, and light micrographs of TS-like cells derived from iPS cells.

FIG. 2 shows the result of a gene expression analysis of TS-like cells derived from iPS cells by introduction of GATA2, GATA3, or TFAP2A gene. The result is presented in a log 2 (expression level) scale after calibrating the expression levels of TS cells and TS-like cells against the expression level of each gene in iPS cells taken as 1. The expression level of each gene in iPS cells is log 2(1)=0.

FIG. 3 shows immunostained images of TS-like cells (ES-TS) derived from ES cells treated with bone morphogenetic protein 4 (BMP4). Antigens against the antibodies used for immunostaining are shown on the left of the pictures.

FIG. 4A shows the result of a comparison of gene expression of TS-like cells (ES-TS; cells derived from ES cells treated with BMP4), TS cells, ES cells, and cells (ES-BMP4) prepared by conventional BMP4 treatment of ES cells.

FIG. 4B shows the result of a comparison of gene expression of TS-like cells (ES-TS; cells derived from ES cells treated with BMP4), TS cells, ES cells, and cells (ES-BMP4) prepared by conventional BMP4 treatment of ES cells.

FIG. 5 shows correlations of gene expression between TS-like cells (ES-TS; cells derived from ES cells treated with BMP4), TS cells, ES cells, and cells (ES-BMP4) prepared by conventional BMP4 treatment of ES cells.

FIG. 6 shows the result of an analysis of DNA methylation pattern of TS-like cells (ES-TS; cells derived from ES cells treated with BMP4), TS cells, ES cells, and cells (ES-BMP4) prepared by conventional BMP4 treatment of ES cells.

FIG. 7 shows the result of an analysis of DNA methylation pattern of ELF5 gene in TS-like cells (ES-TS; cells derived from ES cells treated with BMP4), TS cells, ES cells, and cells (ES-BMP4) prepared by conventional BMP4 treatment of ES cells.

FIG. 8A shows the result of immunostaining with anti-HLA-G antibodies performed for cells differentiated into extravillous cytotrophoblasts (EVTs) from TS-like cells (ES-TS) derived from ES cells treated with BMP4.

FIG. 8B shows the result of a differentiation test for differentiation of TS-like cells (ES-TS; cells derived from ES cells treated with BMP4) and TS cells into extravillous cytotrophoblasts (EVTs). The result is presented as levels of HLA-G expression compared for each cell type, with “ES-TS_EVT” representing cells differentiated into EVTs from TS-like cells, and “TS_EVT” representing cells differentiated into EVTs from TS cells.

FIG. 9A shows the result of immunostaining with anti-hCG antibodies performed for cells differentiated into syncytiotrophoblasts (STs) from TS-like cells (ES-TS) derived from ES cells treated with BMP4.

FIG. 9B shows the result of a differentiation test for differentiation of TS-like cells (ES-TS; cells derived from ES cells treated with BMP4) and TS cells into syncytiotrophoblasts (STs). The result is presented as levels of hCG expression compared for each cell type, with “ES-TS_ST” representing cells differentiated into STs from TS-like cells, and “TS_ST” representing cells differentiated into EVTs from TS cells.

FIG. 10 shows the result of a DNA methylation analysis for DNMT1 and ZFAT in TS-like cells (ES-TS; cells derived from ES cells treated with BMP4), TS cells, ES cells, and cells (ES-BMP4) prepared by conventional BMP4 treatment of ES cells.

FIG. 11 shows the result of a gene expression analysis for BRDT and SOHLH2 in TS-like cells (ES-TS; cells derived from ES cells treated with BMP4), TS cells, and ES cells.

### DESCRIPTION OF EMBODIMENTS

### Definitions

As used herein, the terms “polynucleotide” and “nucleic acid” are interchangeable, and refer to a polymer of nucleotides linked by a phosphodiester bond. The polynucleotide and nucleic acid may be DNA or RNA, or a combination of DNA and RNA. The polynucleotide and nucleic acid may be a polymer of natural nucleotides, or a polymer of natural nucleotides and nonnatural nucleotides (nonnatural nucleotides are, for example, analogs of natural nucleotides, or nucleotides with a modification in at least one of the base, sugar, and phosphate moieties, such as a phosphorothioate backbone), or may be a polymer of nonnatural nucleotides.

In this specification, the sequences of bases in polynucleotides or nucleic acids are presented using the commonly accepted single-letter codes, unless otherwise specifically stated. Base sequences are presented from the 5′ end to the 3′ end, unless otherwise specifically stated.

In this specification, the nucleotide residues constituting polynucleotides or nucleic acids may be presented simply as adenine, thymine, cytosine, guanine, or uracil, or by using their customary single-letter codes.

As used herein, the term “gene” refers to a polynucleotide having at least one open reading frame encoding a specific protein. Genes may include both an exon and an intron.

As used herein, the terms “polypeptide”, “peptide”, and “protein” are interchangeable, and refer to polymers of amino acids joined by the amide bond. The polypeptide, peptide, or protein may be a polymer of natural amino acids, or a polymer of natural amino acids and nonnatural amino acids (nonnatural amino acids are, for example, chemical analogs or modified derivatives of natural amino acids), or may be a polymer of nonnatural amino acids. Amino acid sequences are presented from the N-terminus to the C-terminus, unless otherwise specifically stated.

As used herein, the phrase “to operably link” used in conjunction with polynucleotides means that a first base sequence is disposed close enough to a second base sequence, and that the first base sequence can exert its effects on the second base sequence or on a region placed under the control of the second base sequence. For example, by “to operably link a polynucleotide to a promoter”, it means that a polynucleotide is linked to a promoter, and is expressible under the control of the promoter.

As used herein, the term “expressible state” refers to a state where a polynucleotide is transcribable in a cell to which the polynucleotide is introduced.

As used herein, the term “expression vector” refers to a vector containing a target polynucleotide and having a system by which the target polynucleotide is brought to an expressible state in a cell to which the vector is introduced.

[Cells (TS-Like Cells) Capable of Differentiating into Placenta-Forming Cells]

In an embodiment, there is provided a cell derived from a pluripotent stem cell of a mammal and that is capable of differentiating into a placenta-forming cell, and that is negative for BRDT, and positive for TP63.

The cell of the present embodiment is a TS-like cell having similar characteristics to TS cells. The TS-like cell has the capability to differentiate into placenta-forming cells, as does the TS cell. Examples of the placenta-forming cells include extravillous cytotrophoblasts (EVTs) and syncytiotrophoblasts (STs).

Whether cells have the potential to differentiate into EVTs can be determined by culturing cells under the conditions used to induce differentiation into EVTs, and checking whether the cells have differentiated into EVTs. Examples of conditions that can be used to induce differentiation into EVTs are described in the Examples section below. Specifically, cells can be induced to differentiate into EVTs as follows.

A collagen IV (Col IV)-coated plate containing EVT medium (see Examples) is inoculated with cells, and the cells are cultured after adding Matrigel in 2% of the medium volume. On day 3 after inoculation, the medium is changed to an EVT medium containing no NRG1, and the cells are further cultured after adding Matrigel in 0.5% of the medium volume. On day 6 after inoculation, the medium is changed to an EVT medium containing no NRG1 and KSR, and the cells are further cultured for 6 to 8 days after adding Matrigel in 0.5% of the medium volume.

Whether the cells have differentiated into EVTs after culture performed under these conditions to induce differentiation into EVTs can be confirmed by increased expression levels of HLA-G, aside from morphological observations using a microscope. HLA-G is an EVT marker, and its expression is upregulated by differentiation of TS cells into EVTs. It is accordingly possible to determine differentiation into EVTs when increased expression levels of HLA-G are observed in cultured cells as compared to uncultured cells under the foregoing conditions.

HLA-G (NCBI Gene ID: 3135) may be, for example, one registered with GenBank accession number NM_001363567.1 or NM_002127.5.

Whether cells have the potential to differentiate into STs can be determined by culturing cells under the conditions used to induce differentiation into STs, and checking whether the cells have differentiated into STs. Examples of conditions that can be used to induce differentiation into STs are described in the Examples section below. Specifically, cells can be induced to differentiate into STs as follows.

A collagen IV (Col IV)-coated plate containing ST medium (see Examples) is inoculated with cells, and the cells are cultured. On day 3 after inoculation, the medium is changed to a new ST medium, and the cells are further cultured for 3 days.

Whether the cells have differentiated into STs after culture performed under these conditions to induce differentiation into STs can be confirmed by increased expression levels of CGβ, aside from morphological observations using a microscope. CGβ (or CGB3 (chorionic gonadotropin subunit beta 3) as it is also called) is an ST marker, and its expression is upregulated by differentiation of TS cells into STs. It is accordingly possible to determine differentiation into STs when increased expression levels of CGβ are observed in cultured cells as compared to uncultured cells under the foregoing conditions.

Human CGβ (NCBI Gene ID: 1082) may be, for example, one registered with GenBank accession number NM_000737.3.

The cell of the present embodiment is a cell having similar characteristics to TS cells. However, the cell of the present embodiment is distinguishable from natural TS cells in that the cell of the present embodiment is BRDT negative. BRDT (bromodomain testis associated) is a protein with two bromodomain motifs and a PEST sequence (a cluster of proline, glutamic acid, serine, and threonine residues), characteristics of proteins that undergo rapid degradation. Human BRDT (NCBI Gene ID: 676) may be, for example, one registered with GenBank accession number NM_001242805.2, NM_001242806.2, NM_001242807.2, NM_001242808.2, NM_001242810.2, NM_001726.4, or NM_207189.3.

As used herein, the term “negative” used in conjunction with genes or proteins means that the proteins, or proteins encoded by the genes, are essentially undetectable. Here, “essentially undetectable” means that no detection can be made by ordinary means of protein detection. Such ordinary means of protein detection may be, for example, immunostaining using antibodies against the proteins.

Protein detection may be made by detecting mRNA, and a cell may be determined as being negative for a protein translated from the mRNA when the mRNA is essentially undetectable. For the measurement of mRNA, an ordinary method may be used, for example, such as a method using a next generation sequencer, and a quantitative real-time PCR method. For example, total mRNA extracted from a cell of interest may be analyzed with a next generation sequencer, and the cell can be determined as being negative for the target protein translated from the target mRNA when the target mRNA has a log₂ (FPKM+1) value of less than 1 (preferably less than 0.5, more preferably less than 0.1). FPKM (fragments per kilobase of exon per million reads mapped) is a value that can be obtained using a next generation sequencer, and the FPKM of a transcript t can be determined using the following formula.

FPKM=10⁹×Xₜ/(lₜN),

where Xₜ represents the number of short reads mapped for transcript t, lₜ represents the length (bp) of transcript t, and N represents the total number of short reads mapped.

As used herein, the term “positive” used in conjunction with genes or proteins means that the proteins, or proteins encoded by the genes, are essentially detectable. Here, “essentially detectable” means that detection can be made by ordinary means of protein detection (for example, immunostaining).

As above, a cell may be determined as being positive for a protein translated from corresponding mRNA when the mRNA is detectable by an ordinary method. For example, total mRNA extracted from a cell of interest may be analyzed with a next generation sequencer, and the cell can be determined as being negative for the target protein translated from the target mRNA when the target mRNA has a log₂ (FPKM+1) value of 1 or more (preferably 0.5 or more, more preferably 0.1 or more).

The cell of the present embodiment is a cell derived from a pluripotent stem cell. Here, “pluripotent stem cell” is a cell having pluripotency, and includes embryonic stem cells (ES cells), induced pluripotent stem cells (iPS cells), and pluripotent cells derived from these stem cells. The method of production of pluripotent stem cells is not particularly limited, and a known method may be used. ES or iPS cell lines are also available from cell banks, such as one in Riken BioResource Research Center (RIKEN BRC). Preferably, the pluripotent stem cells are ES cells or iPS cells.

The cell of the present embodiment is a TS-like cell derived by differentiation of a pluripotent stem cell using the method described below in the section [Method of Production of Cell (TS-like Cell) Capable of Differentiating into Placenta-Forming Cell]

The pluripotent stem cell is a mammalian pluripotent stem cell. The mammals are not particularly limited, and may be, for example, primates, rodents, or carnivores. Preferably, the mammals are primates. The primates are, for example, human, chimpanzee, rhesus, or marmoset, preferably human.

Aside from being capable of differentiating into placenta-forming cells, the cell of the present embodiment is positive for TP63, a characteristic similar to TS cells. In contrast, pluripotent stem cells, and TS-like cells induced by traditional methods are negative for TP63. That is, the cell of the present embodiment is similar to TS cells in that it is positive for TP63 as is TS cells, distinguishing itself from pluripotent stem cells and traditional TS-like cells.

Human TP63 (NCBI Gene ID: 8626) may be, for example, one registered with GenBank accession number NM_001114978.1, NM_001114979.1, NM_001114980.1, NM_001114981.1, NM_001114982.1, NM_001329144.1, NM_001329145.1, NM_001329146.1, NM_001329148.1, NM_001329149.1, NM_001329150.1, NM_001329964.1, or NM_003722.5.

Preferably, the cell of the present embodiment is negative for at least one selected from the group consisting of Oct4, Nanog, and Sox2. More preferably, the cell of the present embodiment is negative for at least two of Oct4, Nanog, and Sox2, even more preferably negative for all of Oct4, Nanog, and Sox2. These genes are known undifferentiation markers, and represent positive markers for pluripotent stem cells. Because the cell of the present embodiment is negative for these genes, the cell of the present embodiment is distinguishable from pluripotent stem cells from which the cell of the present embodiment is derived.

Human Oct4 (NCBI Gene ID: 5460) may be, for example, one registered with GenBank accession number NM_001173531.2, NM_001285986.1, NM_001285987.1, NM_002701.6, NM_203289.5, or Z11898.

Human Nanog (NCBI Gene ID: 79923) may be, for example, one registered with GenBank accession number NM_001297698.1, NM_024865.4, or AB093576.

Human Sox2 (NCBI Gene ID: 6657) may be, for example, one registered with GenBank accession number NM_003106.4.

Preferably, the cell of the present embodiment is positive for at least one selected from the group consisting of GATA2, GATA3, and TFAP2A. More preferably, the cell of the present embodiment is positive for at least two of GATA2, GATA3, and TFAP2A, even more preferably positive for all of GATA2, GATA3, and TFAP2A. These genes are TS cell markers. Specifically, being TP63 positive is an indication that the cell of the present embodiment has acquired the same functions possessed by natural TS cells, including the capability to differentiate into EVTs and STs, and that the cell of the present embodiment is distinguishable from pluripotent stem cells from which the cell of the present embodiment is derived.

Human GATA2 (NCBI Gene ID: 2624) may be, for example, one registered with GenBank accession number NM_001145661.1, NM_001145662.1, or NM_032638.4.

Human GATA3 (NCBI Gene ID: 2625) may be, for example, one registered with GenBank accession number NM_001002295.2 or NM_002051.2.

Human TFAP2A (NCBI Gene ID: 7020) may be, for example, one registered with GenBank accession number NM_001032280.2, NM_001042425.1, or NM_003220.3.

Preferably, the cell of the present embodiment is positive for genes such as TFAP2C, in addition to GATA2 and GATA3. TFAP2C is also a TS cell marker.

Human TFAP2C (NCBI Gene ID: 7022) may be, for example, one registered with GenBank accession number NM_003222.4.

Preferably, the cell of the present embodiment is negative for SOHLH2 (or log₂ (FPKM+1)<1).

Human SOHLH2 (NCBI Gene ID: 54937) may be, for example, one registered with GenBank accession number NM_001282147.1 or NM_017826.3.

Preferably, the cell of the present embodiment has about the same fraction of genomic DNA methylation as TS cells. More specifically, the fraction of genomic DNA methylation is preferably 60% or less, more preferably 55% or less, even more preferably 50% or less, particularly preferably 47% or less of the whole genome.

In pluripotent stem cells, the fraction of genomic DNA methylation is about 81% of the whole genome. In TS cells, the fraction of genomic DNA methylation is about 45% of the whole genome. That is, the fraction of genomic DNA methylation can be used as an index of similarity to TS cells.

The lower limit fraction of genomic DNA methylation is, for example, preferably at least 35%, more preferably at least 40% of the whole genome. The preferred range of the fraction of genomic DNA methylation is, for example, 35% to 60%, more preferably 40% to 55%, even more preferably 40% to 47% of the whole genome.

The fraction of genomic DNA methylation in the cell can be measured by using a known method, for example, such as whole genome bisulfite sequencing (WGBS).

In the cell of the present embodiment, the fraction of DNA methylation in the promoter region (chr11: 34513753-34514270; transcription start site: chr11: 34513800)) of ELF5 gene is preferably 5% or less, more preferably 3% or less, even more preferably 2% or less.

In pluripotent stem cells, the fraction of DNA methylation in the promoter region of ELF5 gene is about 76%. In TS cells, the fraction of DNA methylation in the promoter region of ELF5 gene is about 1.7% of the whole genome. That is, the fraction of DNA methylation in the promoter region of ELF5 gene can be used as an index of similarity to TS cells.

The lower limit fraction of DNA methylation in the promoter region of ELF5 gene is, for example, more than 0%, more preferably 0.5% or more, even more preferably 0.8% or more. The preferred range of the fraction of DNA methylation in the promoter region of ELF5 gene is, for example, more than 0% and 5% or less, more preferably 0.5% to 3%, even more preferably 0.8% to 2%.

Human ELF5 (NCBI Gene ID: 2001) may be, for example, one registered with GenBank accession number NM_001243080.1, NM_001243081.1, NM_001422.3, or NM_198381.1.

In the cell of the present embodiment, the fraction of DNA methylation in the promoter region (chr19: 10192830-10195739; transcription start site: chr19: 10195054)) of DNMT1 gene is preferably 20% or less, more preferably 15% or less, even more preferably 10% or less.

In natural TS cells, the fraction of DNA methylation in the promoter region of DNMT1 gene is about 40%. That is, the fraction of DNA methylation in the promoter region of DNMT1 gene can be used as an index that distinguishes natural TS cells from the cell of the present embodiment.

The lower limit fraction of DNA methylation in the promoter region of DNMT1 gene is, for example, 1% or more, more preferably 3% or more, even more preferably 5% or more. The preferred range of the fraction of DNA methylation in the promoter region of DNMT1 gene is, for example, 1% to 20%, more preferably 3% to 20%, even more preferably 5% to 15%, particularly preferably 5% to 10%.

Human DNMT1 (NCBI Gene ID: 1786) may be, for example, one registered with GenBank accession number NM_001130823.3, NM_001318730.1, NM_001318731.1, or NM_001379.3.

In the cell of the present embodiment, the fraction of DNA methylation in the promoter region (chr8: 134694984-134697871; transcription start site: chr8: 134696558)) of ZFAT gene is preferably 50% or more, more preferably 60% or more, even more preferably 70% or more, particularly preferably 80% or more.

In natural TS cells, the fraction of DNA methylation in the promoter region of ZFAT gene is about 40%. That is, the fraction of DNA methylation in the promoter region of ZFAT gene can be used as an index that distinguishes natural TS cells from the cell of the present embodiment.

The upper limit fraction of DNA methylation in the promoter region of ZFAT gene is, for example, 95% or less, more preferably 93% or less. The preferred range of the fraction of DNA methylation in the promoter region of ZFAT gene is, for example, 50% to 95%, more preferably 60% to 95%, even more preferably 70% to 95%, particularly preferably 80% to 93%.

Human ZFAT (NCBI Gene ID: 57623) may be, for example, one registered with GenBank accession number NM_001029939.3, NM_001167583.2, NM_001174157.1, NM_001174158.1, NM_001289394.1, or NM_020863.4.

The fractions of DNA methylation in the promoter regions of ELF5, DNMT1, and ZFAT genes can be measured by using a known method, for example, such as bisulfite sequencing or whole genome bisulfite sequencing (WGBS).

[Method of Production of Cell (TS-Like Cell) Capable of Differentiating into Placenta-Forming Cell]

In an embodiment, the present invention provides a method for producing a cell capable of differentiating into a placenta-forming cell. The cell obtained by the producing method of the present embodiment is the TS-like cell of the embodiment above.

### First Embodiment

The producing method of the present embodiment includes the steps of:

(a) culturing a pluripotent stem cell of a mammal with a medium containing bone morphogenetic protein 4; and

(b) culturing the cell after the step (a) with a medium containing a growth factor and a ROCK inhibitor.

### Step (a)

Step (a) is a step of culturing a pluripotent stem cell of a mammal with a medium containing bone morphogenetic protein 4 (BMP4).

The mammalian pluripotent stem cell used in this step may be any of the cells exemplified in the foregoing section [Cells (TS-Like Cells) Capable of Differentiating into Placenta-Forming Cells]. The mammals are preferably primates, preferably human. The pluripotent stem cell is preferably an ES cell or an iPS cell.

The medium used in this step is a medium containing BMP4. The medium can be prepared by, for example, adding BMP4 to a base medium commonly used for animal cell culture. Examples of the base medium include Doulbecco's modified Eagle's medium (DMEM), DMEM/F12 medium, IMDM medium, Medium 199, Eagle's Minimum Essential medium (EMEM), αMEM medium, Ham's F12 medium, RPMI 1640 medium, Fischer's medium, and a mixed medium of these. The preferred base medium is, for example, DMEM/F12.

BMP4 may be any of various commercially available products.

The BMP4 concentration in the medium is not particularly limited, and may be, for example, 10 to 100 ng/mL, preferably 20 to 80 ng/mL, more preferably 30 to 70 ng/mL, even more preferably 40 to 60 ng/mL.

The base medium may be optionally supplemented with other components, in addition to BMP4. Examples of such other components include one or more serum replacements, for example, such as albumin, transferrin, sodium selenite, ITS-X (Invitrogen), a knockout serum replacement (KSR; an FBS serum replacement for ES cell culture), N2 supplement (Invitrogen), B27 supplement (Invitrogen), fatty acids, insulin, a collagen precursor, trace elements, 2-mercaptoethanol, and 3′-thiolglycerol. Other examples include one or more substances selected from a lipid, an amino acid, L-glutamine, GlutaMax, a non-essential amino acid, a vitamin, a growth factor, an antibiotic, an antioxidizing agent, pyruvic acid, a buffer, and a mineral salt. Preferred examples of the other components include KSR, GlutaMax, non-essential amino acids, 2-mercaptomethanol, and antibiotics (e.g., penicillin, streptomycin).

A specific example of the preferred media is the BMP4 medium described in the Examples section.

The culture temperature in this step may be a temperature commonly used for animal cell culture. The culture temperature may be, for example, 32 to 40° C., and may be appropriately selected in a temperature range of preferably 35 to 38° C.

The CO₂ concentration in a culture is not particularly limited, and is preferably about 2% to 5%, more preferably 5%.

The culture time in this step is not particularly limited, and may be, for example, 1 to 5 days (for example, 24 to 120 hours), preferably 2 to 4 days (for example, 48 to 96 hours), more preferably 3 days (for example, 50 to 80 hours).

In this step, it is preferable to start culture by inoculating a single-cell monolayer of pluripotent stem cells on a plate coated with Matrigel. Preferably, the medium is changed every 20 to 30 hours (for example, every 24 hours).

### Step (b)

Step (b) is a step of culturing the cell after the step (a) with a medium containing a growth factor and a ROCK inhibitor.

The medium used in this step is a medium containing a growth factor and a ROCK inhibitor. The medium may be prepared by, for example, adding a growth factor and a ROCK inhibitor to a base medium commonly used for animal cell culture. The base medium may be any of the base media exemplified for step (a). The preferred base medium is, for example, DMEM/F12.

The growth factor is not particularly limited, and may be, for example, an epidermal growth factor (EGF), insulin, or a transforming growth factor (TGF). These may be commercially available growth factors. The growth factor is preferably an EGF.

The concentration of the growth factor in the medium is not particularly limited, and may be, for example, 10 to 100 ng/mL, preferably 20 to 80 ng/mL, more preferably 30 to 70 ng/mL, even more preferably 40 to 60 ng/mL.

The ROCK (rho associated coiled-coil containing protein kinase, or rho-associated kinase) inhibitor is not particularly limited, as long as it can inhibit the functions of rho-associated kinase. Examples of the ROCK inhibitor include trans-N-(4-pyridyl)-4-(1-aminoethyl)-cyclohexanecarboxamide, 1-(5-isoquinolinylsulfonyl)homopiperazine, and salts thereof. Other examples include low-molecular inhibitors such as Fasudil/HA1077, H-1152, and Wf-536, and derivatives thereof. Still other examples include antisense nucleic acids, siRNAs, and dominant negative mutants against ROCK, and expression vectors therefor.

Examples of commercially available products of trans-N-(4-pyridyl)-4-(1-aminoethyl)-cyclohexanecarboxamide or salts thereof include Y27632 ((R)-(+)-trans-N-(4-pyridyl)-4-(1-aminoethyl)-cyclohexanecarboxamide-2HCl.H₂O). The ROCK inhibitor may be used alone, or two or more ROCK inhibitors may be used in combination.

The ROCK inhibitor is preferably Y27632.

The concentration of the ROCK inhibitor in the medium is not particularly limited, and may be, for example, 0.1 to 50 μM, preferably 1 to 20 μM, more preferably 1 to 10 μM, even more preferably 3 to 8 μM.

The medium used in this step preferably contains at least one selected from the group consisting of an ALK5 inhibitor and a GSK3β inhibitor, in addition to a growth factor and a ROCK inhibitor.

The ALK5 inhibitor is not particularly limited, as long as it can inhibit ALK5 functions. Examples of the ALK5 inhibitor include 4-[4-(1,3-benzodioxol-5-yl)-5-(2-pyridyl)-1H-imidazol-2-yl]benzamide, or salts thereof. Other examples include low-molecular inhibitors such as A83-01 (3-(6-methylpyridin-2-yl)-N-phenyl-4-quinolin-4-ylpyrazole-1-carbothioamide), 2-(3-(6-methylpyridin-2-yl)-1H-pyrazol-4-yl)-1,5-naphthyridine, Wnt3a/BIO, GW788388, SM16, IN-1130, GW6604, SB-505124, and pyrimidine derivatives. Other examples include antisense nucleic acids, siRNAs, and dominant negative mutants against ALK5, and expression vectors therefor. Examples of commercially available products of 4-[4-(1,3-benzodioxol-5-yl)-5-(2-pyridyl)-1H-imidazol-2-yl]benzamide or salts thereof include SB431542. The ALK5 inhibitor may be used alone, or two or more ALK5 inhibitors may be used in combination.

Preferred for use as ALK5 inhibitor is SB431542 or A83-01, more preferably both SB431542 and A83-01.

The concentration of the ALK5 inhibitor in the medium is not particularly limited, and may be, for example, 0.1 to 20 μM, preferably 0.2 to 10 μM, more preferably 0.5 to 5 μM, even more preferably 0.5 to 3 μM. When SB431542 is used as ALK5 inhibitor, the SB431542 concentration in the medium may be, for example, 0.1 to 10 μM, preferably 0.2 to 5 μM, more preferably 0.5 to 3 μM, even more preferably 0.7 to 2 μM. When A83-01 is used as ALK5 inhibitor, the A83-01 concentration in the medium may be, for example, 0.1 to 5 μM, preferably 0.2 to 3 μM, more preferably 0.3 to 2 μM, even more preferably 0.3 to 1 μM.

The GSK3β inhibitor is not particularly limited, as long as it can inhibit GSK3β functions. Examples of the GSK3β inhibitor include 6-[[2-[[4-(2,4-dichlorophenyl)-5-(4-methyl-1H-imidazol-2-yl)-2-pyrimidinyl]amino]ethyl]amino]nicotinonitrile. Other examples include low-molecular inhibitors such as kenpaullone, 1-azakenpaullone, CHIR98014, AR-A014418, CT99021, CT20026, SB216763, AR-A014418, lithium, SB415286, TDZD-8, BIO, BIO-acetoxime, (5-methyl-1H-pyrazol-3-yl)-(2-phenylquinazolin-4-yl)amine, a pyridocarbazole-cyclopentadienyl ruthenium complex, TDZD-8 4-benzyl-2-methyl-1,2,4-thiadiazolidine-3,5-dione, 2-thio(3-iodobenzyl)-5-(1-pyridyl)-[1,3,4]-oxadiazole, OTDZT, alpha-4-dibromoacetophenone, AR-AO 144-18, 3-(1-(3-hydroxypropyl)-1H-pyrrolo[2,3-b]pyridin-3-yl]-4-pyrazin-2-yl-pyrrole-2,5-dione; TWS1 19 pyrrolopyrimidine compounds, L803 H-KEAPPAPPQSpP-NH2, or myristoylated forms of these; 2-chloro-1-(4,5-dibromo-thiophen-2-yl)-ethanone, SB216763, and SB415286. Still other examples include antisense nucleic acids, siRNAs, and dominant negative mutants against GSK3β, and expression vectors therefor. Examples of commercially available products of 6-[[2-[[4-(2,4-dichlorophenyl)-5-(4-methyl-1H-imidazol-2-yl)-2-pyrimidinyl]amino]ethyl]amino]nicotinonitrile include CHIR99021. The GSK3β inhibitor may be used alone, or two or more GSK3β inhibitors may be used in combination.

Preferred for use as GSK3β inhibitor is CHIR99021.

The concentration of the GSK3β inhibitor in the medium is not particularly limited, and may be, for example, 0.1 to 20 μM, preferably 0.2 to 10 μM, more preferably 0.5 to 5 μM, even more preferably 0.5 to 3 μM.

The medium used in this step may be supplemented with a histone deacetylase (HDAC) inhibitor, in addition to the foregoing components.

The HDAC inhibitor is not particularly limited, as long as it can inhibit HDAC functions. Examples of the HDAC inhibitor include low-molecular inhibitors such as valproic acid (VPA), trichostatin A, sodium butyrate, MC1293, and M344. Other examples include antisense nucleic acids, siRNAs, and dominant negative mutants against HDAC, and expression vectors therefor. The HDAC inhibitor may be used alone, or two or more HDAC inhibitors may be used in combination.

Preferred for use as HDAC inhibitor is VPA.

The concentration of the HDAC inhibitor in the medium is not particularly limited, and may be, for example, 0.01 to 10 mM, preferably 0.1 to 5 mM, more preferably 0.5 to 2 mM, even more preferably 0.5 to 1 mM.

The base medium may be optionally supplemented with other components, in addition to the foregoing components. Examples of such other components include sera (e.g., fetal bovine serum (FBS)), and one or more serum replacements, for example, such as albumin, transferrin, sodium selenite, ITS-X (Invitrogen), a knockout serum replacement (KSR; an FBS serum replacement for ES cell culture), N2 supplement (Invitrogen), B27 supplement (Invitrogen), fatty acids, insulin, a collagen precursor, trace elements, 2-mercaptoethanol, and 3′-thiolglycerol. Other examples include one or more substances selected from a lipid, an amino acid, L-glutamine, GlutaMax, a non-essential amino acid, a vitamin, a growth factor, an antibiotic, an antioxidizing agent, pyruvic acid, a buffer, and a mineral salt. Preferred examples of the other components include FBS, bovine serum albumin (BSA), ITS-X, L-ascorbic acid, 2-mercaptomethanol, and antibiotics (e.g., penicillin, streptomycin).

A specific example of the preferred medium is the TS medium described in the Examples section.

The culture temperature in this step may be a temperature commonly used for animal cell culture. The culture temperature may be, for example, 32 to 40° C., and may be appropriately set in a temperature range of preferably 35 to 38° C.

The CO₂ concentration in a culture is not particularly limited, and is preferably about 2% to 5%, more preferably 5%.

The culture time in this step is not particularly limited, and may be, for example, 7 days or longer, preferably 14 days or longer, more preferably 21 days or longer, even more preferably 28 days or longer. The upper limit of culture time is not particularly limited. The TS-like cell can be obtained by culturing the cultured cell after step (a) with the medium used in this step. The TS-like cell obtained can be maintained in an undifferentiated state by subculturing the as-cultured TS-like cell with the medium used in this step.

In this step, it is preferable to start culture by inoculating the cell after step (a) on a plate coated with collagen IV (Col IV). Preferably, the cell is subcultured every week (every 7 days). The cell after step (a) may be released from the culture plate with trypsin or a trypsin replacement (e.g., TrypLE), and may be inoculated to a culture plate containing the medium used in this step.

### Second Embodiment

The producing method of the present embodiment includes the steps of:

(a′) introducing at least one gene selected from the group consisting of GATA2, GATA3, and TFAP2A to a pluripotent stem cell of a mammal; and

(b) culturing the cell after the step (a′) with a medium containing a growth factor and a ROCK inhibitor.

Step (a′)

Step (a′) is a step of introducing at least one gene selected from the group consisting of GATA2, GATA3, and TFAP2A to a pluripotent stem cell of a mammal.

The mammalian pluripotent stem cell used in this step may be any of the cells exemplified in the foregoing section [Cells (TS-Like Cells) Capable of Differentiating into Placenta-Forming Cells]. The mammals are preferably primates, preferably human. The pluripotent stem cell is preferably an ES cell or an iPS cell.

The gene introduced into a mammalian pluripotent stem cell is at least one gene selected from the group consisting of GATA2, GATA3, and TFAP2A. These genes may be introduced alone, or two or more of these genes may be introduced in combination. Preferably, any one of GATA2, GATA3, and TFAP2A is introduced.

The organism from which the GATA2, GATA3, and TFAP2A genes are derived is preferably the same organism from which the pluripotent stem cell is derived. For example, when using human pluripotent stem cells, it is preferable to use human GATA2, GATA3, and TFAP2A genes. The human GATA2, GATA3, and TFAP2A genes may have, for example, the base sequences presented in the foregoing section [Cells (TS-Like Cells) Capable of Differentiating into Placenta-Forming Cells]. Information of base sequences of corresponding genes from other mammals is available from known database, such as the GenBank sequence database.

The GATA2, GATA3, and TFAP2A genes are not limited to the wild-type genes, and may include a mutation (deletion, substitution, insertion, or addition), provided that the GATA2, GATA3, and TFAP2A genes with such mutations have inductive capability for TS-like cells. Here, “inductive capability for TS-like cells” means the function to induce differentiation of pluripotent stem cells into TS-like cells that come to have the properties described in the foregoing section [Cells (TS-Like Cells) Capable of Differentiating into Placenta-Forming Cells] after step (b) following introduction of the genes into pluripotent stem cells.

Examples of GATA2, GATA3, or TFAP2A gene that can be used in this step include the genes listed in (A) to (G) below.

(A) Wild-type GATA2, GATA3, or TFAP2A gene (e.g., a polynucleotide consisting of the nucleotide sequence represented by SEQ ID NO: 3 (GATA2), SEQ ID NO: 1 (GATA3), or SEQ ID NO: 5 (TFAP2A).

(B) A polynucleotide consisting of a nucleotide sequence that codes for wild-type GATA2, GATA3, or TFAP2A protein (e.g., a protein consisting of the amino acid sequence represented by SEQ ID NO: 4 (GATA2), SEQ ID NO: 2 (GATA3), or SEQ ID NO: 6 (TFAP2A)).

(C) A polynucleotide that codes for a protein consisting of the amino acid sequence of wild-type GATA2, GATA3, or TFAP2A protein (e.g., the amino acid sequence represented by SEQ ID NO: 4 (GATA2), SEQ ID NO: 2 (GATA3), or SEQ ID NO: 6 (TFAP2A)) with mutation of one or a plurality of amino acids, and that has inductive capability for TS-like cells.

(D) A polynucleotide that codes for a protein consisting of an amino acid sequence with at least 70% sequence identity with the amino acid sequence of wild-type GATA2, GATA3, or TFAP2A protein (e.g., the amino acid sequence represented by SEQ ID NO: 4 (GATA2), SEQ ID NO: 2 (GATA3), or SEQ ID NO: 6 (TFAP2A)), and that has inductive capability for TS-like cells.

(E) A polynucleotide consisting of a nucleotide sequence with mutation in one or a plurality of the nucleotides of wild-type GATA2, GATA3, or TFAP2A gene (e.g., a polynucleotide consisting of the nucleotide sequence represented by SEQ ID NO: 3 (GATA2), SEQ ID NO: 1 (GATA3), or SEQ ID NO: 5 (TFAP2A)), and that has inductive capability for TS-like cells.

(F) A polynucleotide consisting of a nucleotide sequence with at least 70% sequence identity with wild-type GATA2, GATA3, or TFAP2A gene (e.g., a polynucleotide consisting of the nucleotide sequence represented by SEQ ID NO: 3 (GATA2), SEQ ID NO: 1 (GATA3), or SEQ ID NO: 5(TFAP2A)), and that has inductive capability for TS-like cells.

(G) A polynucleotide that hybridizes under stringent conditions with wild-type GATA2, GATA3, or TFAP2A gene (e.g., a polynucleotide consisting of the nucleotide sequence represented by SEQ ID NO: 3 (GATA2), SEQ ID NO: 1 (GATA3), or SEQ ID NO: 5 (TFAP2A)), and that has inductive capability for TS-like cells.

In (C) and (E) above, “mutation” may be any of deletion, substitution, addition, and insertion, or a combination of these.

In (C) above, the number of “plurality” is not particularly limited, and may be, for example, 2 to 30, preferably 2 to 20, more preferably 2 to 10, even more preferably 2 to 5, particularly preferably 2 or 3.

In (E) above, the number of “plurality” is not particularly limited, and may be, for example, 2 to 60, preferably 2 to 50, more preferably 2 to 40 or 2 to 30, even more preferably 2 to 20 or 2 to 10, particularly preferably 2 to 5, or 2 or 3.

In (D) and (F) above, the sequence identity is not particularly limited, as long as it is at least 70%. However, the sequence identity is preferably at least 80%, more preferably at least 85%, even more preferably at least 95%. The sequence identity between amino acid sequences or between nucleotide sequences is determined as a percentage of matching amino acids or matching nucleotides relative to the whole amino acid sequence or nucleotide sequence, excluding gaps in the alignment, when two amino acid sequences or nucleotide sequences are paired to create the greatest number of matches for corresponding amino acids or corresponding nucleotides with gaps interposed in positions equivalent of insertions and deletions. The sequence identity between amino acid sequences or between nucleotide sequences can be determined using various types of homology search software known in the art. For example, the sequence identity between amino acid sequences or between nucleotide sequences can be obtained by calculations based on an alignment obtained by the known homology search software BLASTP.

In (G) above, the stringent conditions are, for example, conditions described in Molecular Cloning—A LABORATORY MANUAL THIRD EDITION (Sambrook et al., Cold Spring Harbor Laboratory Press). As an example, polynucleotides are hybridized by being incubated for several hours to overnight at 42 to 70° C. in a hybridization buffer consisting of 6×SSC (a 20×SSC composition: a 3 M sodium chloride and 0.3 M citric acid solution, pH 7.0), a 5×Denhardt's solution (100×Denhardt's solution composition: 2 mass % bovine serum albumin, 2 mass % Ficoll, 2 mass % polyvinylpyrrolidone), 0.5 mass % SDS, 0.1 mg/mL salmon sperm DNA, and 50% formamide. The wash buffer used for washing after incubation is, for example, preferably a 0.1 mass % SDS-containing 1×SSC solution, more preferably a 0.1 mass % SDS-containing 0.1×SSC solution.

In (B) to (E) above, the degenerate codons are preferably codons with high frequencies of usage in pluripotent stem cells of the mammal used. For example, for human pluripotent stem cells, it is preferable to use codons with high frequencies of usage in human cells. That is, it is preferable to optimize codons for human.

The method used to introduce the genes into a pluripotent stem cell is not particularly limited, and a known method may be used. For example, the genes may be introduced into the target pluripotent stem cell by being cloned into an expression vector that can be expressed in the target pluripotent stem cell.

In addition to the genes, the expression vector preferably includes a promoter that regulates expression of the genes. In such an expression vector, the promoter is disposed upstream of the genes to regulate expression of the genes, and the genes are operably linked to the promoter.

Examples of the promoter include the SRα promoter, the SV40 early promoter, a LTR of a retrovirus, the CMV (cytomegalovirus) promoter, the RSV (Rous sarcomavirus) promoter, the HSV-TK (herps simplex virus thymidine kinase) promoter, the EF1α promoter, a metallothionein promoter, and a heat shock promoter. The promoter may be used with an enhancer for the IE gene of human CMV. As an example, it is possible to use the CAG promoter (containing a cytomegalovirus enhancer, a chicken β-actin promoter, and a polyA signal site of β-globin gene).

In addition to promoters, the expression vector may contain, for example, an enhancer, a polyA addition signal, a marker gene, a replication initiation site, and a gene that codes for a protein that regulates replication by binding to the replication initiation site, as desired. The marker gene refers to a gene that enables screening or selection of cells by being introduced into cells. Specific examples of the marker gene include a drug resistance gene, a fluorescence protein gene, a luminescent enzyme gene, and a chromogenic enzyme gene. These may be used alone, or two or more of these may be used in combination. Specific examples of the drug resistance gene include a neomycin resistance gene, a tetracycline resistance gene, a kanamycin resistance gene, a zeocin resistance gene, a hygromycin resistance gene, and a puromycin resistance gene. Specific examples of the fluorescence protein gene include a green fluorescence protein (GFP) gene, a yellow fluorescence protein (YFP) gene, and a red fluorescence protein (RFP) gene. Specific examples of the luminescent enzyme gene include a luciferase gene. Specific examples of the chromogenic enzyme gene include a β-galactosidase gene, a β-glucuronidase gene, and an alkali phosphatase gene.

The type of expression vector is not particularly limited, and known expression vectors may be used. Examples of expression vectors include episomal vectors, artificial chromosome vectors, plasmid vectors, and virus vectors.

Examples of the episomal vectors include vectors in which a sequence necessary for autonomous replication, such as those originating in EBV and SV40, is contained as a vector element. Specific examples of such vector elements necessary for autonomous replication include a replication initiation site, and a gene that codes for a protein that regulates replication by binding to the replication initiation site. Examples are replication initiation site oriP and EBNA-1 gene in the case of EBV, and replication initiation site on and SV40LT gene in the case of SV40.

Examples of the artificial chromosome vectors include a YAC (yeast artificial chromosome) vector, a BAC (bacterial artificial chromosome) vector, and a PAC (P1-derived artificial chromosome) vector.

The plasmid vectors are not particularly limited, as long as the vectors can be expressed in a pluripotent stem cell to which the vectors are introduced. Examples of plasmid vectors for expression in animal cells include pA1-11, pXT1, pRc/CMV, pRc/RSV, and pcDNAI/Neo.

Examples of the virus vectors include retrovirus vectors (including lentivirus vectors), adenovirus vectors, adeno-associated virus vectors, Sendai virus vectors, herpes virus vectors, vaccinia virus vectors, poxvirus vectors, poliovirus vectors, sylbisvirus vectors, rhabdovirus vectors, paramyxovirus vectors, and orthomyxovirus vectors.

The method used to introduce an expression vector into a pluripotent stem cell is not particularly limited, and may be appropriately selected according to the type of expression vector. Examples of methods that can be used to introduce an expression vector into a pluripotent stem cell include the lipofection method, the microinjection method, the DEAE dextran method, the gene-gun technique, the electroporation method, and the calcium phosphate method. When the expression vector is a virus vector, for example, the polybrene technique may be used for infection of a pluripotent stem cell with the virus vector.

When the expression vector containing the genes is carrying an antibiotic resistance gene as a selectable marker, cells after the introduction of the expression vector can be efficiently selected by culturing the cells with a medium containing an antibiotic corresponding to the antibiotic resistance gene after the expression vector is introduced.

### Step (b)

Step (b) is as described in step (b) of First Embodiment.

The producing method of the present embodiment enables a pluripotent stem cell to be induced to differentiate into a TS-like cell having the capability to differentiate into a placenta-forming cell. The TS-like cell obtain by the producing method of the present embodiment can be used as a research material for basic research, such as in studies of early development of mammals, and functional analyses of the placenta. The TS-like cell also can be used for an understanding of the pathogenesis of disorders associated with abnormal placenta, or for the development of therapeutic methods for such disorders. Other applicable areas include reproductive medicine and regenerative medicine.

### EXAMPLES

The following describes the present invention through Examples. It is to be noted, however, that the present invention is not limited to the descriptions of the following Examples.

### [Materials and Methods]

### (TS Cell Culture)

A TS cell line was cultured following the previously described method (Okae, H., Toh, H., Sato, T. et al. Cell Stem Cell 22, 50-63 e56, doi:10.1016/j.stem.2017.11.004 (2018)). The TS cell line was cultured in a TS medium containing 5 μg/mL Col IV (354233; Corning, Corning, N.Y.). The cells were cultured in 5% CO₂ at 37° C., and the culture medium was changed after 2 days.

The TS medium was prepared by adding the following components to DMEM/F12 (048-29785; Fujifilm Wako, Osaka, Japan).

0.1 mM 2-Mercaptoethanol (21985023; Thermo Fisher Scientific, Waltham, Mass.)

0.2% FBS (16141-079; Thermo Fisher Scientific)

0.5% Penicillin-Streptomycin (15140122; Thermo Fisher Scientific)

0.3% BSA (017-22231; Fujifilm Wako)

1% ITS-X supplement (094-06761; Fujifilm Wako)

1.5 μg/ml L-Ascorbic acid (013-12061; Fujifilm Wako)

50 ng/mL EGF (053-07871; Fujifilm Wako)

2 μM CHIR99021 (038-23101; Fujifilm Wako)

0.5 μM A83-01 (035-24113; Fujifilm Wako)

1 μM SB431542 (031-24291; Fujifilm Wako)

0.8 mM VPA (227-01071; Fujifilm Wako)

5 μM Y-27632 (036-24023; Fujifilm Wako)

### (Culture of ES Cells and iPS Cells)

A human ES cell line (SEES 1, 4, 6) and an iPS cell line (Nips-B2) were cultured on a Matrigel (354234; Corning) in an ES medium [hPSC medium delta (197-17571; Fujifilm Wako) supplemented with 35 ng/ml FGF2 (064-05381; Fujifilm Wako) and 10 μM Y-27632 (Fujifilm Wako)], following the manufacturer's protocol. The cells were subcultured after dissociating the colonies with TrypLE expression enzyme (12604021; Thermo Fisher Scientific) by allowing the enzyme to act at 37° C. for 5 minutes. The cells were cultured in 5% CO₂ at 37° C. On the following day, the medium was changed to an ES medium containing no Y-27632, and, subsequently, this medium was used daily as a new medium.

(Induced Differentiation into TS-Like Cells Via BMP4 Signaling)

Single monolayer ES cells or iPS cells were inoculated on a Matrigel-coated plate containing ES medium (10,000 cells/cm²), and cultured in 5% CO; at 37° C. On the following day, the medium was changed to a BMP4 medium (Krendl, C., Shaposhnikov, D., Rishko, V. et al. Proc Natl Acad Sci USA 114, E9579-E9588, doi:10.1073/pnas.1708341114 (2017)). A fresh BMP4 medium was used every 24 hours. On day 3, the cells were dissociated with TrypLE (Thermo Fisher Scientific) by allowing the enzyme to act at 37° C. for 13 to 15 minutes. At a split ratio of 1:2, the cells were inoculated on a Col IV-coated plate containing a TS medium, and cultured in 5% CO₂ at 37° C. before subculturing the cells every week. The ES cells or iPS cells after BMP4 treatment were a heterogeneous population after the first passage. However, TS-like colonies, capable of self replication, appeared after 4 passages. Thereafter, the ES cells or iPS cells after BMP4 treatment were regularly subcultured every week at a 1:5 to 1:10 split ratio.

The BMP4 medium was prepared by adding the following components to DMEM/F12 (048-29785; Fujifilm Wako, Osaka, Japan).

20% Knockout serum replacement (1082802 8; Thermo Fisher Scientific)

1% GlutaMax (35050038; Thermo Fisher Scientific)

1% Nonessential amino acids (1140050; Thermo Fisher Scientific)

0.1 mM 2-Mercaptoethanol (21985023; Thermo Fisher Scientific)

1% Penicillin-Streptomycin (Thermo Fisher Scientific)

50 ng/mL BMP4 (020-18851; Fujifilm Wako)

(Induced Differentiation into TS-Like Cells by Gene Introduction)

cDNA was cloned into a pLVSIN-EF1α base transfer vector, using an In-Fusion® HD cloning kit (Takara Bio, Shiga, Japan). After codon optimization, cDNA of GATA2, GATA3, or TFAP2A (GATA2: SEQ ID NO: 1, 2; GATA3: SEQ ID NO: 3, 4; TFAP2A: SEQ ID NO: 5, 6) was synthesized in pLVSIN-EF1α vector, and transferred to a lentivector selectable by puromycin. Single monolayer iPS cells were inoculated on a Matrigel-coated plate containing an ES medium (100,000 cells/cm²). The medium was then changed to a lentivirus stock supplemented with 6 μg/mL polybrene (Sigma-Aldrich, St-Louis, Mo.). After 24 hours from infection, the cells were cultured for 2 days in 0.5 to 1.0 μg/mL puromycin for selection. After selection, the iPS cells were inoculated at a 1:10 split ratio to a Col IV-coated plate containing a TS medium, and cultured in 5% CO₂ at 37° C. before subculturing the cells every week. iPS cells that incorporated the gene were a heterogeneous population after the first passage. However, TS-like colonies, capable of self replication, appeared after 4 passages. Thereafter, the iPS cells that had incorporated the gene were regularly subcultured every week at a 1:5 to 1:10 split ratio.

The primers (5′-3′) used for cloning of GATA2, GATA3, and TFAP2A are as follows. Here and below, “F” means a forward primer, and “R” a reverse primer.

GATA3_F:
(SEQ ID NO: 7)
GCTAAACGACCCCTCCAAGATA
GATA3_R:
(SEQ ID NO: 8)
TCATGCCTTACAGCTACCCAGA
GATA2_F:
(SEQ ID NO: 9)
TCTGCACCCAGACCCTGA
GATA2_R:
(SEQ ID NO: 10)
GGAGTGGTGTCGGCCTTC
TFAP2A_F:
(SEQ ID NO: 11)
AGAGCCGCGATGTCCATACT
TFAP2A_R:
(SEQ ID NO: 12)
AGCAGTAGCAGCAGCAGGAAG

(Differentiation into EVTs or STs)

Methods for induced differentiation into extravillous cytotrophoblasts (EVTs) and syncytiotrophoblasts (STs) are previously described (Okae, H., Toh, H., Sato, T. et al. Cell Stem Cell 22, 50-63 e56, doi:10.1016/j.stem.2017.11.004 (2018)). In order to induce TS cells or TS-like cells to differentiate into EVT cells, TS cells and TS-like cells were inoculated on a 1 μg/mL Col IV-coated plate (Corning) containing an EVT medium, at a density of 8,000 cells/cm². After the inoculation of the cells to the medium, a Matrigel (Corning) was added in 2% of the medium volume. On day 3 after inoculation, the medium was changed to an EVT medium containing no NRG1 (Cell Signaling), and a Matrigel (Corning) was added in 0.5% of the medium volume. On day 6 after inoculation, the medium was changed to an EVT medium containing no NRG1 (Cell Signaling) and KSR (Thermo Fisher Scientific), and a Matrigel (Corning) was added in 0.5% of the medium volume. The cells were analyzed after 6 to 8 days from the last medium change.

The EVT medium was prepared by adding the following components to DMEM/F12 (048-29785; Fujifilm Wako, Osaka, Japan).

0.5% Penicillin-Streptomycin (Thermo Fisher Scientific)

0.3% BSA (Fujifilm Wako)

1% ITS-X supplement (Fujifilm Wako)

100 ng/ml NRG1 (5218SC; Cell Signaling)

7.5 μM A83-01 (Fujifilm Wako)

2.5 μM Y27632 (Fujifilm Wako)

4% KSR (Thermo Fisher Scientific)

In order to induce differentiation of TS cells or TS-like cells to ST cells, TS cells and TS-like cells were inoculated on a 2.5 μg/mL Col IV-coated plate (Corning) containing an ST medium, at a density of 10,000 cells/cm². The medium was changed on day 3 after inoculation, and the cells were analyzed on day 6 after inoculation.

The ST medium was prepared by adding the following components to DMEM/F12 (048-29785; Fujifilm Wako, Osaka, Japan).

0.1 mM 2-Mercaptoethanol (Thermo Fisher Scientific)

0.5% Penicillin-Streptomycin (Thermo Fisher Scientific)

0.3% BSA (Fujifilm Wako)

1% ITS-X supplement (Fujifilm Wako)

2.5 μM Y27632 (Fujifilm Wako)

2 μM Forskolin (Fujifilm Wako)

4% KSR (Thermo Fisher Scientific)

### (RNA Sequencing)

Total RNA was extracted with an RNeasy® Mini Kit and an RNase-Free DNase (QIAGEN, Valencia, Calif.), and was used for library construction with a TruSeq® Stranded mRNA LT Sample Prep Kit (Illumina, San Diego, Calif.) following the manufacturer's protocol. The integrity of RNA was evaluated using a TapeStation 2200 (Agilent Technologies, Santa Clara, Calif.). All samples had RINe values of more than 9. The library was sequenced with 101 bp paired-end reads using an Illumina HiSeq 2500 Platform (Illumina). Reads were aligned against reference genome (UCSC hg38) using a TopHat with a Refseq gene annotation (Trapnell, C., Roberts, A., Goff, L. et al. Nat Protoc 7, 562-578, doi:10.1038/nprot.2012.016 (2012)). For female samples, the Y chromosome was excluded from reference genome. The expression levels (FPKM) of Refseq gene were calculated with Cufflinks (Trapnell, C., Roberts, A., Goff, L. et al. Nat Protoc 7, 562-578, doi:10.1038/nprot.2012.016 (2012)). X-linked genes and Y-linked genes in the pseudoautosomal region were excluded from analysis.

(Whole-Genome Bisulfite Sequencing (WGBS)) WGBS was performed by post-bisulfite adaptor tagging (PBAT) (Miura, F., Enomoto, Y., Dairiki, R. et al. Nucleic Acids Res 40, e136, doi:10.1093/nar/gks454 (2012)). In brief, genomic DNA was purified by phenol/chloroform extraction and ethanol precipitation. Genomic DNA with 0.5% (w/w) unmethylated a phage DNA (Promega, Madison, Wis.) was used to prepare a library, following the PBAT protocol. The concentration of the PBAT product was quantified with a KAPA Library Quantification Kit for Illumina platforms (KAPA Biosystems, Woburn, Mass.). The PBAT library was sequenced with 101 bp single-end reads using an Illumina HiSeq 1500 equipped with HCS v2.0.5 and RTA v1.17.20 (Illumina). Reads were aligned against reference genome using a Bismark (Krueger, F. & Andrews, S. R. Bismark: Bioinformatics 27, 1571-1572, doi:10.1093/bioinformatics/btr167 (2011)). The methylation level (UCSC hg38) of each cytosine was calculated using a Bismark Methylation Extractor. For calculations of methylation level at each CpG site, reads from both strands were combined. The methylation level of CpG covered by at least 5 reads was analyzed.

### (Quantitative Real-Time PCR Analysis)

Total RNA was prepared with an RNeasy Mini Kit and an RNase-Free DNase (QIAGEN). First-strand cDNA was synthesized from the total RNA using a PrimeScript® II (Takara Bio), and a real-time PCR reaction was performed using a StepOnePlus Real-Time PCR System (Applied Biosystems, Foster City, Calif.) and an SYBR Premix Ex Taq II (Takara Bio). The level of target mRNA was determined by using the ΔΔCt method with a GAPDH used as an internal standard.

The primers (5′-3′) used for quantitative real-time PCR analysis are as follows.

OCT4_F:
(SEQ ID NO: 13)
GCTCGAGAAGGATGTGGTCC
OCT4_R:
(SEQ ID NO: 14)
CGTTGTGCATAGTCGCTGCT
NANOG_F:
(SEQ ID NO: 15)
GCAGAAGGCCTCAGCACCTA
NANOG_R:
(SEQ ID NO: 16)
AGGTTCCCAGTCGGGTTCA
GATA2_F:
(SEQ ID NO: 17)
TCAAGCCCAAGCGAAGAC
GATA2_R:
(SEQ ID NO: 18)
CACAGGCGTTGCAGACAG
GATA3_F:
(SEQ ID NO: 19)
CTCATTAAGCCCAAGCGAAG
GATA3_R:
(SEQ ID NO: 20)
TCTGACAGTTCGCACAGGAC
TFAP2A_F:
(SEQ ID NO: 21)
AACATGCTCCTGGCTACAAAA
TFAP2A_R:
(SEQ ID NO: 22)
AGGGGAGATCGGTCCTCA
TP63_F:
(SEQ ID NO: 23)
AGAAACGAAGATCCCCAGATGA
TP63_R:
(SEQ ID NO: 24)
CTGTTGCTGTTGCCTGTACGTT

### (Bisulfite Sequencing)

Genomic DNA was purified by phenol/chloroform extraction and ethanol precipitation. An EZ DNA Methylation-Gold Kit (Zymo Research, Orange, Calif.) was used for treatment of genomic DNA with bisulfite. A TaKaRa EpiTaq HS (Takara Bio) was used for PCR. PCR products were purified, and cloned into a pGEM-T vector (Promega). Sequencing of individual clones was performed by Eurofins Genomics (Tokyo, Japan). On average, 20 clones were sequenced for each sample.

The primers (5′-3′) used for bisulfite sequencing are as follows.

ELF5_BSF:
(SEQ ID NO: 25)
TGATGGATATTGAATTTGAATTTAAAGGTA
ELF5_BSR:
(SEQ ID NO: 26)
CAATAAAAATAAAACACCTATAACCTTAT

### (Immunostaining)

Cells were fixed with 4% paraformaldehyde (Fujifilm Wako) for 10 minutes, and was subjected to 0.3% Triton X-100 (Fujifilm Wako) for 5 minutes for permeabilization, before blocking with 2% FBS/PBS at room temperature for 1 hour. The cells were then incubated overnight at 4° C. with primary antibodies. Alexa Fluor 488 or Alexa Fluor 555-binding secondary antibodies (Cell Signaling) were used as secondary antibodies. The nucleus was stained with Hoechst 33258, and photographed with a fluorescence microscope (BZ-X710, Keyence, Osaka, Japan).

The primary antibodies used are as follows.

PE-conjugated anti-HLA-G (Novus Biologicals, Littleton, Colo.; 1:100 dilution),

PE-conjugated anti-SDC1 (#130-119-840, Miltenyi Biotec, Bergisch Gladbach, Germany; 1:100 dilution)

Anti-GATA3 (#5852, Cell Signaling, Danvers, Mass.; 1:100 dilution)

Anti-TFAP2C (#sc-12762, Santa Cruz Biotechnology, Dallas, Tex.; 1:100 dilution)

Anti-hCG (#IS508, Dako, Hamburg, Germany; 1:10 dilution)

Anti-OCT4 (#4439, Cell Signaling; 1:100 dilution)

### (Creation of Graphs)

The methylation level of CpG cytosine was visualized with Integrative Genomics Viewer (IGV) software (www.broadinstitute.org/igv/). Heat maps were crated using the heatmap.2 function of the gplots package. Bar graphs were created using the ggplot2 package available from R (www.R-project.org/).

### [Results]

<TS-Like Cells Derived from iPS Cells>
(Preparation of TS-Like Cells from iPS Cells)

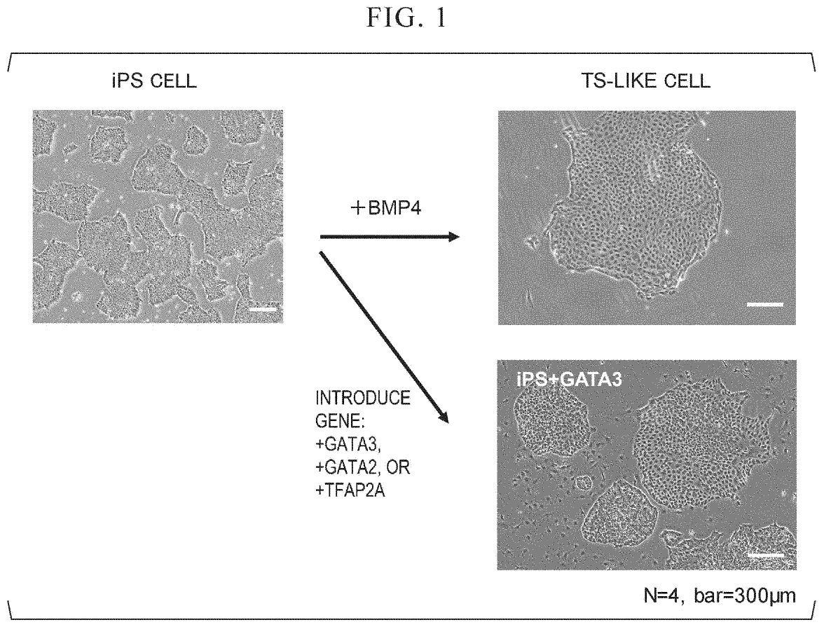

iPS cells were induced to differentiate into TS-like cells following the method described in the foregoing section (Induced Differentiation into TS-Like Cells via BMP4 Signaling). This produced TS-like cells similar in morphology to TS cells (see FIG. 1).

Separately, iPS cells were induced to differentiate into TS cells following the method described in the foregoing section (Induced Differentiation into TS-Like Cells by Gene Introduction). This produced TS-like cells similar in morphology to TS cells, irrespective of which of the GATA3, GATA2, and TFAP2A genes (cDNAs) was introduced (see FIG. 1). As a representative example, FIG. 1 shows TS-like cells obtained after introduction of GATA3 gene into iPS cells.

### (Gene Expression Analysis)

Gene expression in TS-like cells obtained after introduction of GATA2, GATA3, or TFAP2A gene into iPS cells was analyzed, and the result was compared with that from TS cells.

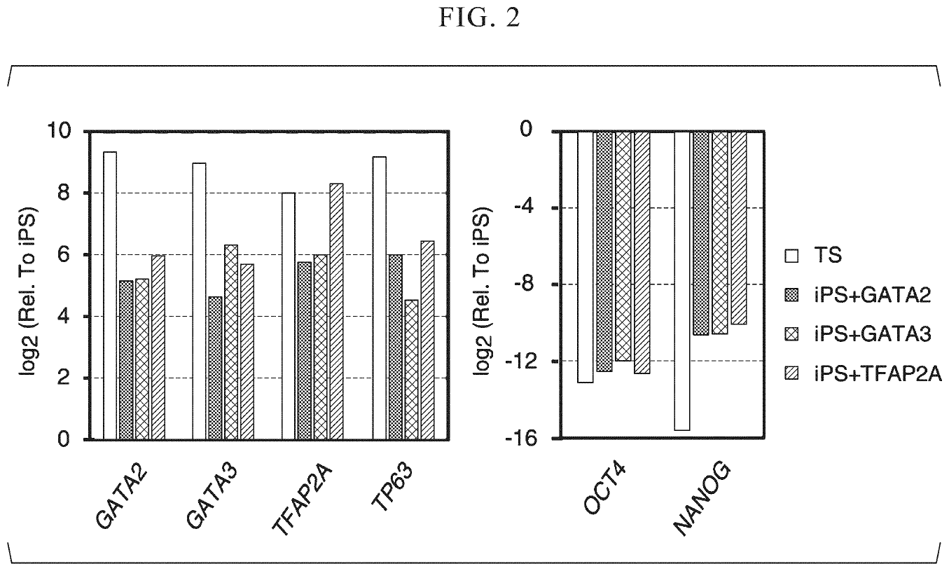

The result of comparison is presented in FIG. 2. FIG. 2 shows expression levels relative to the expression level of iPS cells taken as log 2(1). As shown in FIG. 2, the TS-like cells obtained after gene introduction had similar gene expression to TS cells, regardless of the gene. The TS-like cells were also similar to TS cells in that these cells both had reduced expression of OCT4 and NANOG, marker genes of iPS cells.

<TS-Like Cells Derived from ES Cells>
(Preparation of TS-Like Cells from ES Cells)

ES cells were induced to differentiate into TS-like cells following the method described in the foregoing section (Induced Differentiation into TS-Like Cells via BMP4 Signaling). This produced TS-like cells similar in morphology to TS cells.

### (Immunostaining)

Anti-OCT4 antibodies (anti-OCT4), anti-TFAP2C antibodies (anti-TFAP2C), and anti-GATA3 antibodies (anti-GATA3) were used for immunostaining of TS-like cells, ES cells, and TS cells.

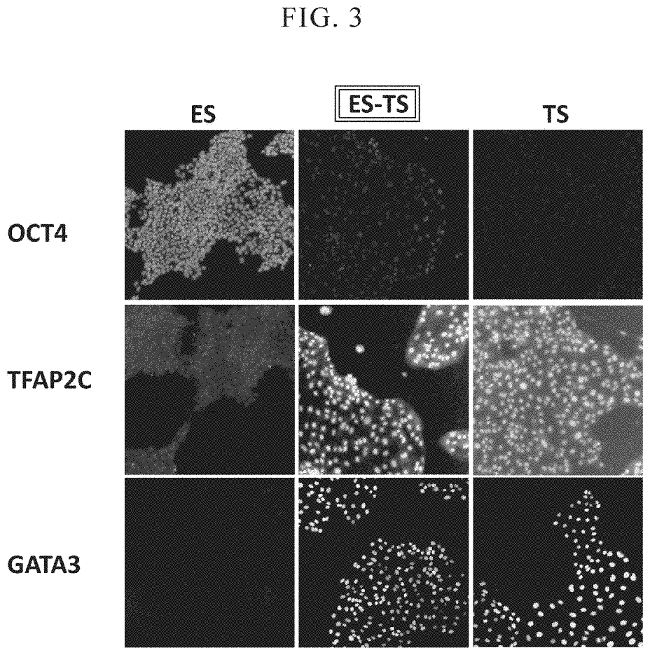

The results are presented in FIG. 3. In FIG. 3 and subsequent figures, “ES-TS” represents TS-like cells derived from ES cells. As shown in FIG. 3, the TS-like cells derived from ES cells had gene expression patterns that were more similar to TS cells than to ES cells.

### (Gene Expression Analysis)

Gene expression of TS-like cells was analyzed, and the result was compared with results from ES cells, BMP4-treated cells (cells after a BMP4 treatment of ES cells using a conventional technique; cultured in BMP4 medium for 72 hours), and TS cells.

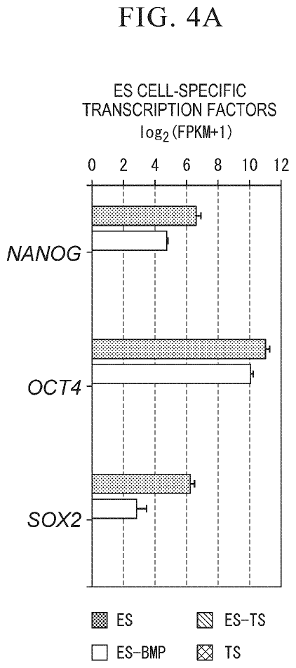

The results are presented in FIG. 4A and FIG. 4B. In FIGS. 4A and 4B and subsequent figures, “ES-BMP4” represents cells after a BMP4 treatment of ES cells using a conventional technique. As shown in FIGS. 4A and 4B, the TS-like cells derived from ES cells had gene expression patterns that were more similar to TS cells than to ES cells. Specifically, the TS-like cells were negative for expression of NANOG, OCT4, and SOX2, and positive for expression of GATA3, GATA2, TFAP2C, TFAP2A, and TP63.

In contrast, the BMP4-treated cells prepared by using a conventional technique still showed high expression of transcription factors specific to ES cells. The BMP4-treated cells also had low expression of transcription factors specific to TS cells, compared to the TS-like cells and ES cells. The result for TP63, expressed in TS cells and TS-like cells, was negative in the BMP4-treated cells prepared by using a conventional technique, as in ES cells.

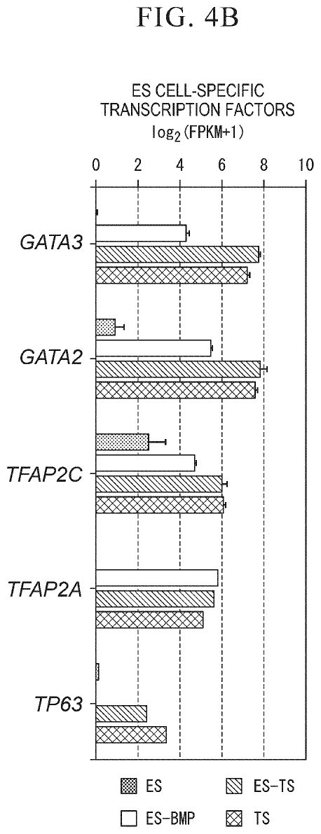

FIG. 5 is a diagram representing correlations of gene expression between TS cells, TS-like cells (ES-TS), ES cells, and ES cells subjected to a BMP4 treatment using a conventional technique (ES-BMP4 cells). It can be seen that TS-like cells and TS cells have a high correlation. The BMP4-treated cells prepared by using a conventional technique had a higher correlation with ES cells than with TS cells.

### (Analysis of DNA Methylation Pattern)

The DNA methylation pattern of TS-like cells was analyzed, and the result was compared with results from ES cells, BMP4-treated cells (cells after a BMP4 treatment of ES cells using a conventional technique), and TS cells.

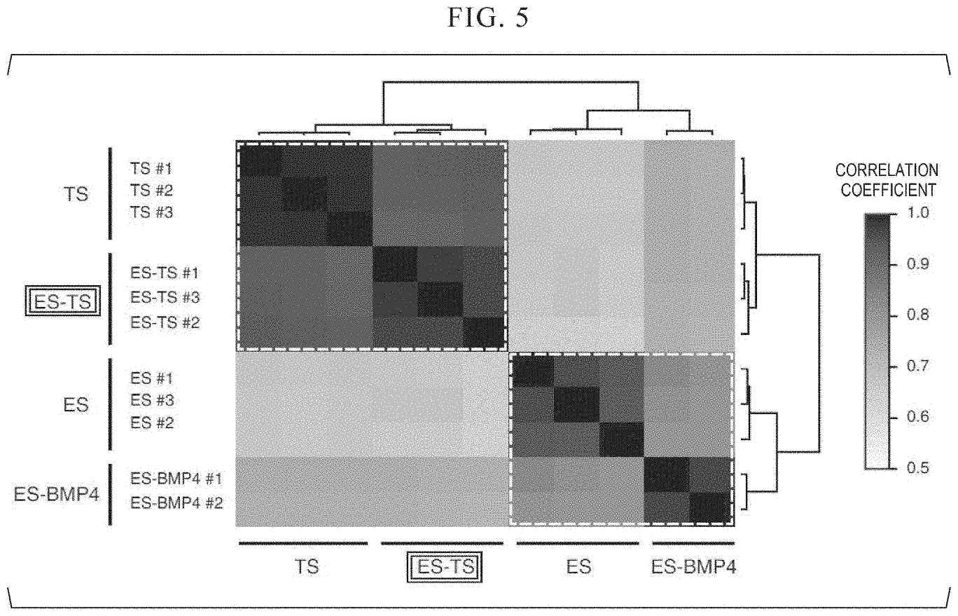

The result of comparison is presented in FIG. 6. FIG. 6 shows analysis results for a 10 Mb region in chromosome 21. As shown in FIG. 6, the TS-like cells derived from ES cells had a DNA methylation pattern that was more similar to TS cells than to ES cells. In contrast to the ES cells in which the percentage of methylation in the whole genome was about 81%, the percentage of methylation in TS-like cells was reduced to about 45%, about the same as the result observed for TS cells.

The BMP4-treated cells prepared by using a conventional technique had a methylation pattern similar to that of ES cells.

### (DNA Methylation Analysis for ELF5)

A DNA methylation analysis was performed for TS-like cells, ES cells, and TS cells in the promoter region (chr11: 34513753-34514270 (transcription start site: chr11: 34513800)) of ELF5 gene. ELF5 is a gene that has been reported to show low methylation in the placenta (Hemberger M, et al., Hum Mol Genet. 2010 Jun. 15; 19(12):2456-67).

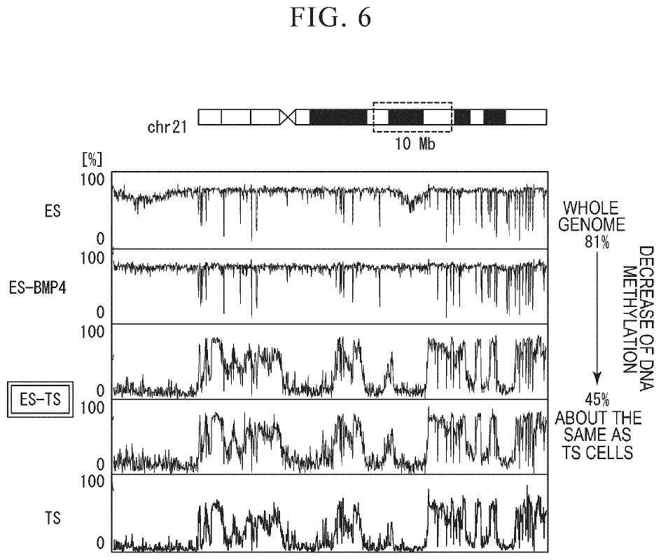

The results are presented in FIG. 7. As shown in FIG. 7, the TS-like cells had a percentage methylation of 1.0%, similar to the percentage of methylation observed for the TS cells.

A previous study reports that the percentage of ELF5 methylation in cells prepared by a conventional BMP4 treatment of ES cells is 29.5% (Lee C Q, et al., Stem Cell Rep. 2016 6:257-272). It therefore can be seen that, with regard to a fraction of DNA methylation in ELF5, the TS-like cells are far more similar to TS cells than BMP4-treated cells prepared by using a conventional technique.

### (Differentiation Test for TS-Like Cells)

TS-like cells and TS cells were tested to differentiate into EVTs or STs, following the method described in the foregoing section (Differentiation into EVTs or STs). The differentiated cells were analyzed for expression of HLA-G and hCG, which are markers of EVT and ST, respectively.

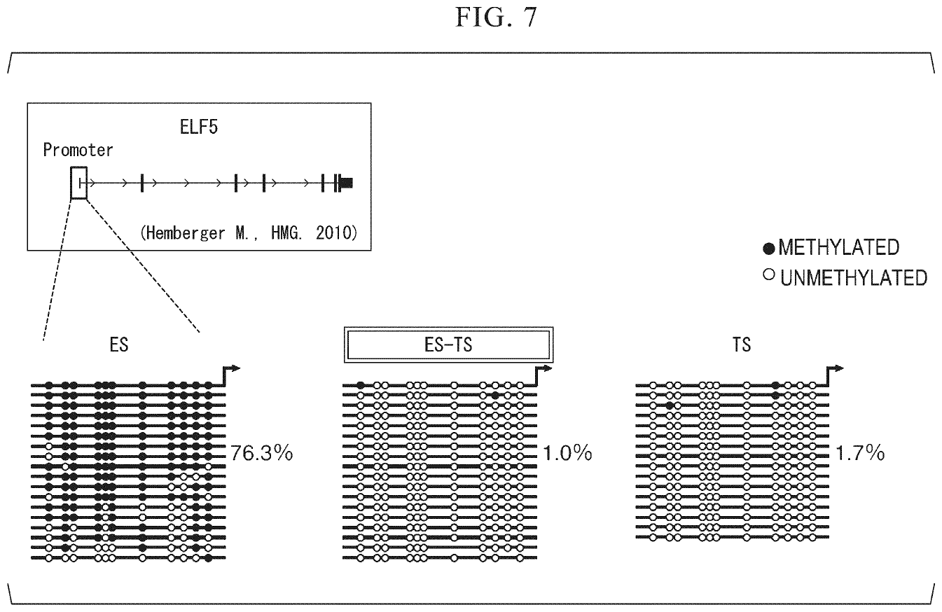

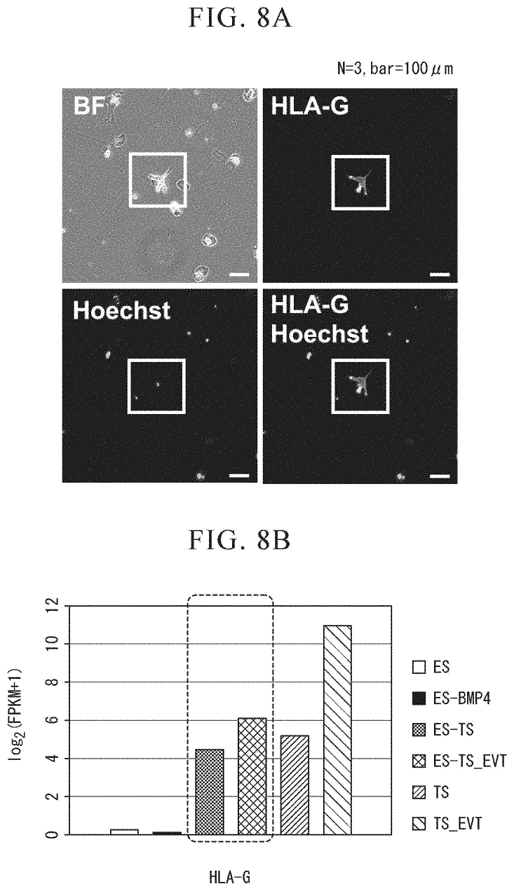

The results of differentiation test for EVT are presented in FIG. 8A and FIG. 8B. FIG. 8A shows the result of immunostaining with anti-HLA-G antibodies (PE-conjugated anti-HLA-G) performed for cells differentiated into EVTs from TS-like cells. FIG. 8B shows the result of comparison of HLA-G expression levels between each cell type. In FIG. 8B (and FIG. 9B), “ES-TS_EVT” represents cells differentiated into EVTs from TS-like cells, and “TS_EVT” represents cells differentiated into EVTs from TS cells.

As shown in FIGS. 8A and 8B, the cells differentiated into EVTs from TS-like cells had increased expression of HLA-G, as in the cells differentiated into EVTs from TS cells. This result confirmed that the TS-like cells have the potential to differentiate into EVTs, as does the TS cells.

The results of differentiation test for ST are presented in FIG. 9A and FIG. 9B. FIG. 9A shows the result of immunostaining with anti-hCG antibodies (anti-hCG) performed for cells differentiated into STs from TS-like cells. FIG. 9B shows the result of comparison of hCG expression levels between each cell type.

As shown in FIGS. 9A and 9B, the cells differentiated into STs from TS-like cells had increased expression of hCG, as in the cells differentiated into STs from TS cells. This result confirmed that the TS-like cells have the potential to differentiate into STs, as does the TS cells.

### (DNA Methylation Analysis for DNMT1 and ZFAT)

A DNA methylation analysis was performed for TS-like cells, ES cells, and TS cells in the promoter region of DNMT1 gene (transcription start site of DNMT1 gene (chr19: 10192830-10195739; transcription start site: chr19: 10195054)) and the promoter region of ZFAT gene (chr8: 134694984-134697871; transcription start site: chr8: 134696558)). These regions are known to show placenta-specific methylation (germline differentially methylated regions: gDMRs).

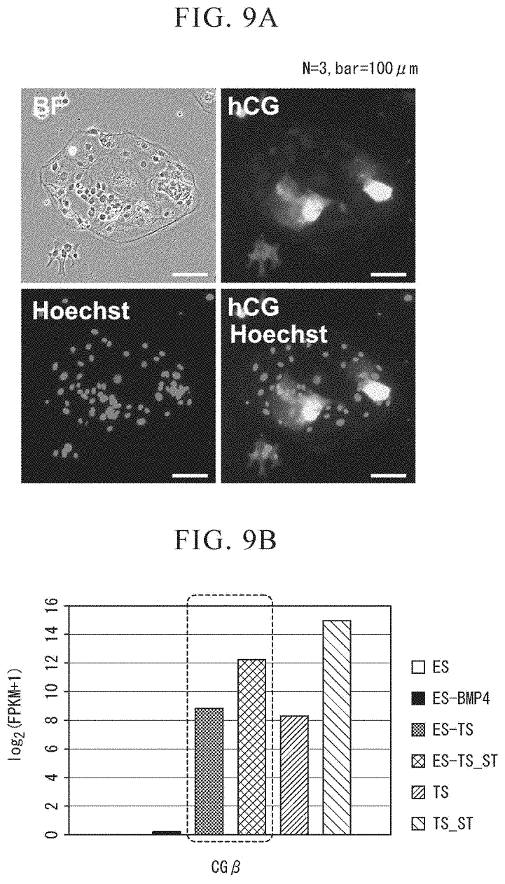

The results are presented in FIG. 10. As shown in FIG. 10, the TS-like cells had a lower percentage of methylation than the TS cells in DMNT1 gene. The TS-like cells had a higher percentage of methylation than the TS cells in ZFAT gene. These results indicate that the TS-like cells have different characteristics from TS cells with regard to the methylation patterns of these genes.

### (Gene Expression Analysis for BRDT and SOHLH2)

A gene expression analysis was performed for BRDT gene and SOHLH2 gene in TS-like cells, TS cells, and ES cells.

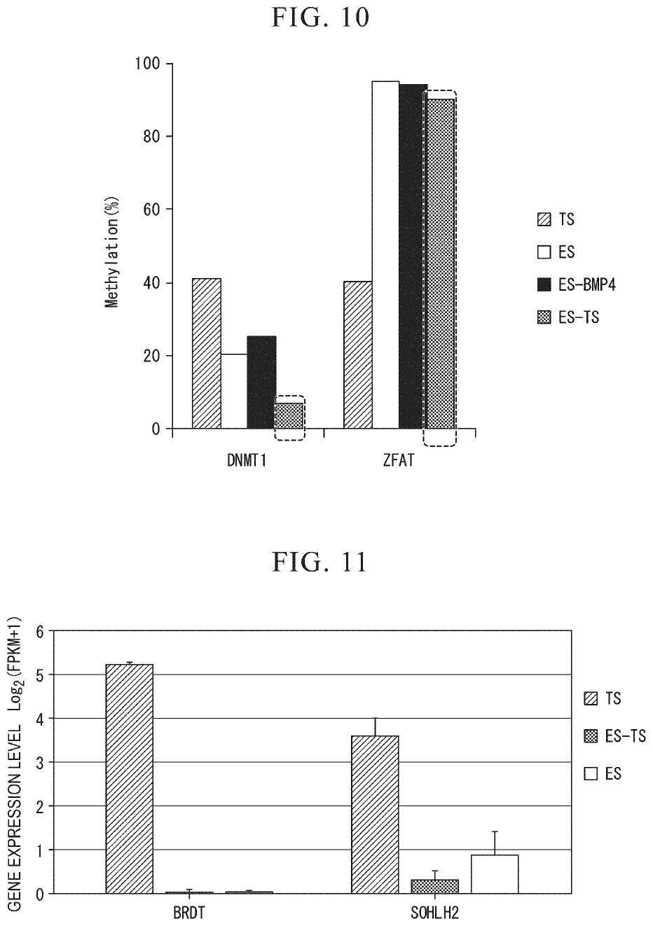

The result is presented in FIG. 11. As shown in FIG. 11, the TS-like cells, unlike TS cells, showed hardly any expression of BRDT and SOHLH2 genes. This result indicates that the TS-like cells have different characteristics from TS cells with regard to the expression of these genes.

### INDUSTRIAL APPLICABILITY

According to the present invention, a method for producing a TS-like cell from a pluripotent stem cell is provided. The present invention also provides a TS-like cell produced by the method. TS-like cells provided by the present invention are applicable to studies of early development of mammals, and functional analyses of the placenta. Other applicable areas include regenerative medicine.

1. A cell derived from a pluripotent stem cell of a mammal and that is capable of differentiating into a placenta-forming cell, and that is negative for BRDT, and positive for TP63.

2. The cell according to claim 1, which is negative for at least one selected from the group consisting of Oct4, Nanog, and Sox2.

3. The cell according to claim 1, which is positive for at least one selected from the group consisting of GATA2, GATA3, and TFAP2A.

4. The cell according to claim 1, wherein a fraction of methylation of genomic DNA is 60% or less of whole genome.

5. The cell according to claim 1, wherein the placenta-forming cell is an extravillous cytotrophoblast or a syncytiotrophoblast.

6. A method for producing a cell capable of differentiating into a placenta-forming cell,
the method comprising:
(a) culturing a pluripotent stem cell of a mammal with a medium containing bone morphogenetic protein 4; and
(b) culturing the cell after (a) with a medium containing a growth factor and a ROCK inhibitor.

7. A method for producing a cell capable of differentiating into a placenta-forming cell,
the method comprising:
(a′) introducing at least one gene selected from the group consisting of GATA2, GATA3, and TFAP2A to a pluripotent stem cell of a mammal; and
(b) culturing the cell after (a′) with a medium containing a growth factor and a ROCK inhibitor.

8. The method for producing a cell capable of differentiating into a placenta-forming cell according to claim 6, wherein the medium in (b) further contains at least one selected from the group consisting of an ALK5 inhibitor and a GSK3β inhibitor.

9. The method for producing a cell capable of differentiating into a placenta-forming cell according to claim 7, wherein the medium in (b) further contains at least one selected from the group consisting of an ALK5 inhibitor and a GSK3β inhibitor.


SEQUENCE LISTING (26 sequences)

SEQ ID NO: 1 - DNA, 1890 bp, Homo sapiens
  CDS (211)..(1545)
gctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatga

SEQ ID NO: 2 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 3 - DNA, 1568 bp, Homo sapiens
  CDS (35)..(1477)
tctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcc

SEQ ID NO: 4 - protein, 480 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 5 - DNA, 1420 bp, Homo sapiens
  CDS (10)..(1311)
agagccgcgatgtccatacttgccaaaatgggggactggcaggaccgtcacgacggcaccagcaacgggacggcacggttgccccagctgggcactgtaggtcaatctccctacacgagcgccccgccgctgtcccacacccccaatgccgacttccagcccccatacttccccccaccctaccagcctatctacccccagtcgcaagatccttactcccacgtcaacgacccctacagcctgaaccccctgcacgcccagccgcagccgcagcacccaggctggcccggccagaggcagagccaggagtctgggctcctgcacacgcaccgggggctgcctcaccagctgtcgggcctggatcctcgcagggactacaggcggcacgaggacctcctgcacggcccacacgcgctcagctcaggactcggagacctctcgatccactccttacctcacgccatcgaggaggtcccgcatgtagaagacccgggtattaacatcccagatcaaactgtaattaagaaaggccccgtgtccctgtccaagtccaacagcaatgccgtctccgccatccctattaacaaggacaacctcttcggcggcgtggtgaaccccaacgaagtcttctgttcagttccgggtcgcctctcgctcctcagctccacctcgaagtacaaggtcacggtggcggaagtgcagcggcggctctcaccacccgagtgtctcaacgcgtcgctgctgggcggagtgctccggagggcgaagtctaaaaatggaggaagatctttaagagaaaaactggacaaaataggattaaatctgcctgcagggagacgtaaagctgccaacgttaccctgctcacatcactagtagagggagaagctgtccacctagccagggactttgggtacgtgtgcgaaaccgaatttcctgccaaagcagtagctgaatttctcaaccgacaacattccgatcccaatgagcaagtgacaagaaaaaacatgctcctggctacaaaacagatatgcaaagagttcaccgacctgctggctcaggaccgatctcccctggggaactcacggcccaaccccatcctggagcccggcatccagagctgcttgacccacttcaacctcatctcccacggcttcggcagccccgcggtgtgtgccgcggtcacggccctgcagaactatctcaccgaggccctcaaggccatggacaaaatgtacctcagcaacaaccccaacagccacacggacaacaacgccaaaagcagtgacaaagaggagaagcacagaaagtgaggctctcctcccgccccgcccctcccacgcctcaccagccccccgcgcgcccaccctccggcgggtgacagctccgggatcagcaacccttcctgctgctgctactgct

SEQ ID NO: 6 - protein, 433 aa, Homo sapiens
MSILAKMGDWQDRHDGTSNGTARLPQLGTVGQSPYTSAPPLSHTPNADFQPPYFPPPYQPIYPQSQDPYSHVNDPYSLNPLHAQPQPQHPGWPGQRQSQESGLLHTHRGLPHQLSGLDPRRDYRRHEDLLHGPHALSSGLGDLSIHSLPHAIEEVPHVEDPGINIPDQTVIKKGPVSLSKSNSNAVSAIPINKDNLFGGVVNPNEVFCSVPGRLSLLSSTSKYKVTVAEVQRRLSPPECLNASLLGGVLRRAKSKNGGRSLREKLDKIGLNLPAGRRKAANVTLLTSLVEGEAVHLARDFGYVCETEFPAKAVAEFLNRQHSDPNEQVTRKNMLLATKQICKEFTDLLAQDRSPLGNSRPNPILEPGIQSCLTHFNLISHGFGSPAVCAAVTALQNYLTEALKAMDKMYLSNNPNSHTDNNAKSSDKEEKHRK

SEQ ID NO: 7 - DNA, 22 bp, Artificial sequence
  GATA3_F1 primer
gctaaacgacccctccaagata

SEQ ID NO: 8 - DNA, 22 bp, Artificial sequence
  GATA3_RP primer
tcatgccttacagctacccaga

SEQ ID NO: 9 - DNA, 18 bp, Artificial sequence
  GATA2_F1 primer
tctgcacccagaccctga

SEQ ID NO: 10 - DNA, 18 bp, Artificial sequence
  GATA2_R1 primer
ggagtggtgtcggccttc

SEQ ID NO: 11 - DNA, 20 bp, Artificial sequence
  TFAP2A_F1 primer
agagccgcgatgtccatact

SEQ ID NO: 12 - DNA, 21 bp, Artificial sequence
  TFAP2A_R1 primer
agcagtagcagcagcaggaag

SEQ ID NO: 13 - DNA, 20 bp, Artificial sequence
  OCT4_F1 primer
gctcgagaaggatgtggtcc

SEQ ID NO: 14 - DNA, 20 bp, Artificial sequence
  OCT4_R1 primer
cgttgtgcatagtcgctgct

SEQ ID NO: 15 - DNA, 20 bp, Artificial sequence
  NANOG_F1 primer
gcagaaggcctcagcaccta

SEQ ID NO: 16 - DNA, 19 bp, Artificial sequence
  NANOG_R1 primer
aggttcccagtcgggttca

SEQ ID NO: 17 - DNA, 18 bp, Artificial sequence
  GATA2_F2 primer
tcaagcccaagcgaagac

SEQ ID NO: 18 - DNA, 18 bp, Artificial sequence
  GATA2_R2 primer
cacaggcgttgcagacag

SEQ ID NO: 19 - DNA, 20 bp, Artificial sequence
  GATA3_F2 primer
ctcattaagcccaagcgaag

SEQ ID NO: 20 - DNA, 20 bp, Artificial sequence
  GATA3_R2 primer
tctgacagttcgcacaggac

SEQ ID NO: 21 - DNA, 21 bp, Artificial sequence
  TFAP2A_F2 primer
aacatgctcctggctacaaaa

SEQ ID NO: 22 - DNA, 18 bp, Artificial sequence
  TFAP2A_R2 primer
aggggagatcggtcctga

SEQ ID NO: 23 - DNA, 22 bp, Artificial sequence
  TP63_F1 primer
agaaacgaagatccccagatga

SEQ ID NO: 24 - DNA, 22 bp, Artificial sequence
  TP63_R1 primer
ctgttgctgttgcctgtacgtt

SEQ ID NO: 25 - DNA, 30 bp, Artificial sequence
  ELF5_BSF1 primer
tgatggatattgaatttgaatttaaaggta

SEQ ID NO: 26 - DNA, 30 bp, Artificial sequence
  ELF5_BSR1 primer
caataaaaataaaaacacctataaccttat